(19) United States
(12) Patent Application Publication
(10) Pub. No.: US 2020/0367483 A1
(43) Pub. Date: Nov. 26, 2020

(54) METHOD AND SYSTEM FOR RECORDING AND/OR MONITORING POPULATIONS OF INSECTS

(71) Applicant: BIOGENTS AKTIENGESELLSCHAFT, Regensburg (DE)

(72) Inventors: Martin GEIER, Sulzbach-Rosenberg (DE); Michael WEBER-GRABAU, Sunnyvale, CA (US)

(73) Assignee: Biogents AG, Regensburg (DE)

(21) Appl. No.: 16/634,260

(22) PCT Filed: Jul. 25, 2019
(86) PCT No.: PCT/EP2018/070179

§ 371 Date: Jan. 27, 2020

(30) Foreign Application Priority Data
Jul. 28, 2017 (DE) 10 2017 213 076.8

(51) Int. Cl. A01M 1/02; G01N 21/64; G01N 23/00; G01N 29/14; A01K 11/00; A01K 67/033; A01M 1/14

(52) U.S. Cl. CPC A01M 1/026; G01N 21/6428; G01N 23/00; G01N 2021/6439; A01K 11/00; A01K 67/0333; A01M 1/14; G01N 29/14

(57) ABSTRACT

Disclosed are a system (10) and a method used for the detection and/or monitoring of insect populations (16), in which insects (16) of at least one defined genus are attracted, at least temporarily retained within a defined compartment (12), and sensed and/or analyzed with regard to definable features (14). The invention comprises: features or method steps, as appropriate:
attracting the defined genus of insects (16) within a definable spatial vicinity (20) with an attracting stimuli (22);
transferring the attracted insects (16) into a defined compartment interior (12) while preventing, at least for a definable time interval, an inadvertent escape, at least of a large number of the insects (16);
detecting specific features (14) of the insects (16) with a sensor unit (26);
transmitting sensor signals (30) supplied by the sensor unit (26) to an electronic analysis device and/or evaluation device (28) disposed downstream from the sensor unit (26).

### CLAIM OF PRIORITY

The present application claims priority to International Application PCT/EP2018/070179, filed Jul. 25, 2018, which in turn claims priority to German Application 10 2017 213 076.8, filed Jul. 28, 2017, which are incorporated by reference.

### FIELD OF THE INVENTION

The present invention relates to a method used for the detection and/or monitoring of insect populations. The invention moreover relates to an insect detection system and/or insect monitoring system used for the detection and/or monitoring of insects (whether flying or not)of at least one defined genus, which insects are attracted and at least temporarily retained within a defined compartment, and which are sensed and/or analyzed with regard to definable features.

### BACKGROUND OF THE INVENTION

Numerous different methods are available and applicable for the monitoring of the insect control measures as well as for the verification of the effectiveness of the measures. There is an overall, worldwide increasing interest in methods for insect population control and, in particular, in methods to reduce existing populations of pest insects and populations that are known to transmit diseases. One possibility to reduce and repress such populations lies in releasing sterile insects within existing populations, which is also known as so-called “sterile insect technology” (SIT). Particular attention is paid in this context to a certain species of mosquitoes, the Aedes Aegypti, which is to be regarded as a particularly problematic species in spreading the Zika virus in currently prevalent epidemics, but which is also held responsible for dengue epidemics, for spreading yellow fever and other diseases.

SIT, as it is already being used in some areas, is based on releasing sterile male mosquitoes that have been bred in laboratories. These male insects of the species to be repressed are released in large quantities so that they can mate with the wild female insects and, in particular, compete with the wild male population of the insects. As a result of this technology, the population affected in such a manner is reduced, and thereby also the risk of the diseases being transmitted.

In a different but, in principle, similar approach, the wild insect populations are infected with a bacterium called Wolbachia, which causes a reduction of the capability to transmit dengue fever in the insects infected in such a way.

All of these comparable approaches have in common that they require extensive monitoring of the male and female mosquitoes of the affected populations with the intention to reliably identify their relevant attributes. When applying SIT, for example, it is desirable to be able to distinguish released male insects that were bred in the laboratory from the wild male insects; this can be made possible, for instance, by markers based on fluorescent dust, which allows the previously released male insects to be counted after capturing them again later. The currently available methods applied in practice for such a monitoring and detection of released male insects from laboratory populations are based on a manual count and require high monitoring and laboratory efforts.

Different approaches to the detection and monitoring of insect populations are known from the prior art. For example, WO 2012 054 397 A1 discloses an insect monitoring device with a lure for attracting insects and with one or more sensors for the detection of insects, in particular, of a certain target insect species. The provided sensors can be, for example, bio-impedance sensors, optical sensors, such as IR sensors, ultrasonic sensors, or the like. In some embodiments, the optical sensors can serve to distinguish the species. Counting the insects is also proposed. The sensor signals can subsequently be collected in a network, processed, and recorded.

WO 2012 054 990 A1 discloses a device for the real-time detection of insects. The device comprises an open inspection cylinder with a camera system for real-time insect monitoring and data collection. The camera system is intended to enable obtaining high-quality images of the appropriate insects and counting the insects. Inside the cylinder, the insects can moreover be detected by an infrared detector.

WO 2016 064 735 A1 discloses a wireless sensor system for determining the growth of mosquito populations and for data acquisition and data analysis. The sensor system is intended to enable detecting, monitoring, and controlling the mosquito population. The number of mosquitoes can be recorded through appropriate counting systems. In addition, it is possible to take into account further data, such as pressure, temperature, humidity, movement, and time, etc. These data are transmitted to a computer unit and correspondingly processed there.

WO 2016 168 347 A1 describes a mosquito trap and a network and method of detecting, counting, trapping, and discarding mosquitoes and/or insects. The device comprises, among others, one or more sound capture devices, video capture devices, and detectors, in each instance configured to detect the species and sex of the particular mosquito and insect. Other sensors, such as IR sensors, proximity sensors, and ultrasonic sensors can be provided to increase the detection accuracy for mosquitoes/insects. The mosquitoes/insects are attracted by a so-called attracting device, which may be designed in various ways.

EP 2 149 301 B1 discloses an automated determining of the presence of insects on a catch plate. A camera directed toward the surface of the catch plate is provided for this purpose. The camera is intended to enable determining the number and species of the insects, for example, using vision technologies. The camera can preferably be formed by a scanner. It is moreover possible to examine the insects and, for example, to make them identifiable by color. The collected data allow predictions on the development of a population.

US 2015 023 40 49 A1 moreover discloses a detection system for objects, such as insects, which system comprises an enclosure that can be illuminated and which is equipped with a sensor that senses disturbances in light intensity so that the system can detect the presence of moving objects inside the enclosure. The measuring principle is based on the fact that moving objects, such as flying insects, have different impacts on the light detected by the sensor, such as absorption of the light, changing shadows, and other effects, which can be sensor-detected. The sensor data can be evaluated to acquire information on the presence and/or type of the detected insects inside the enclosure.

The use of such systems not only allows remote monitoring in real time, but, according to the signal processing, moreover allows recording and storing the detected sensor values in order to perform, for example, an analysis of the processes that can be carried out continuously or at cyclic intervals.

A primary object of the present invention therefore lies in developing an effective method that makes it possible to automatically count insects, such as mosquitoes, where the method should not only comprise attracting male and female insects, but also identifying insect species or insect genera, distinguishing between male and female insects, recognizing insects or mosquitoes bearing, for example, a defined attribute or a defined marker, determining insect density or mosquito density in a definable area, transmitting the data acquired in such a way to a remote data collection center and/or data processing center, and processing and evaluating these data.

This object of the invention is achieved by the subject matter of the independent claims. Features of advantageous developments of the invention are set forth in the dependent claims.

### SUMMARY OF THE INVENTION

In order to achieve the above-mentioned object, the invention proposes a method used for the detection and/or monitoring of insect populations, in which insects of at least one defined genus are attracted, at least temporarily retained within a defined compartment, and sensed and/or analyzed with the method having the features of the independent method claim. The method serves, in particular, for the detection and/or monitoring of populations of flying insects of a defined genus, such as gnats, mosquitoes such as Aedes aegypti, or other species or genera, by the insects being attracted, at least temporarily retained within a defined compartment, and sensed and/or analyzed with regard to definable features. The attracting implies bringing the insects into the range of the defined compartment by the insects being attracted there. In the method according to the invention, the defined species or genus of insects is attracted into the range of or within a definable spatial vicinity by attracting stimuli that are attractive to the insects or flying insects, whereupon the attracted insects are transferred into a defined compartment interior, in particular into an interior space of an insect trap, while preventing, at least for a definable time interval, an inadvertent escape, at least of a large number of the insects having been transferred into the defined compartment interior. The transfer of the insects into the defined compartment interior and/or the retention of the insects there can be carried out in different ways, for example, by a suction device and/or by elements of a suction trap.

The method furthermore provides the detecting of specific features of the insects located within the defined compartment interior or within the insect trap by a sensor unit suitable or equipped therefor as well as the providing of the specific features of the insects detected by the sensor unit and the transmitting of the sensor signals supplied by the sensor unit to an electronic analysis device and/or evaluation device disposed downstream from the sensor unit.

It can be provided in the method, for example, that the sex (m/f) and/or the species or genus of the insects are detected as specific features of the insects located within the defined compartment interior or within the insect trap. As only the female insects reproduce, however, the eggs laid by the female insects have to be fertilized by male insects, it has proved an effective measure for insect population control to produce a competition situation in such a way that large quantities of sterile male insects of the species or genus to be repressed are released and then compete with the wild male insects such that the lack of fertilization of the female insects gradually leads to fewer insects being able to reproduce, with the result that an overall reduction of the populations can be achieved. If the success of such measures is to be verified, monitored, and controlled, it is necessary to be able to distinguish the male insects from the female insects of the species or genus to be monitored.

For this purpose, the method can provide, for example, that the specific features of the insects located within the defined compartment interior or within the insect trap are formed by markers adhering to the insects and/or by markers having been applied to them by stamping. If sterile male insects are released within a certain environment, it is expedient to provide these insects with a marker, which can be identified and sensed with the aid of the monitoring method according to the invention. Such largely unlosable markers are a valuable distinctive feature between insects bred in the laboratory and released afterward and wild insects of the same kind, which could otherwise hardly be distinguished in a reliable manner.

The markers can be formed, for example, by genetic modifications to the insects or flying insects, which genetic modifications are detected by the sensor technology. Optionally, the markers can also be formed by radioactive markers, which are detected by the sensor technology. It is likewise conceivable that the markers are formed by optical and/or fluorescent markers, which are detected by the sensor technology. Suitable for this purpose are, for example, fluorescent substances, such as fluorescent dust, which adheres to the insects and which even in smallest quantities can be identified by the sensor unit employed . Preferably, such markers are used for the method according to the invention that are formed by features and/or by markers imprinted onto the detected and/or released populations, where the markers can be detected by the sensor unit with sufficient reliability such that only, for example, the released male, sterile insects are provided with the specific markers to allow a later identification of released insects and their distinction from insects of an insect population already present within the monitored compartment.

A further expedient variant of an optical marker and/or a marker that is sensor-detectable in another manner can be formed by using, for example, rhodamine B. If the rhodamine B has been administered to the insects to be sensed by suitable feeding, for example, by mixing rhodamine B in low concentration into suitable foods, such as sugar solution or honey solutions, the fluorescent dye can be effectively detected on the monitored insects by a suitable sensor unit.

Combinations of such markers are also possible in order to improve the reliability of the identification. When using an appropriately sensitive sensor unit, even the smallest traces of such markers can be adequate to allow a distinction of the insects marked in such a way from wild insects.

An advantageous variant of the method according to the invention can provide that the sensor unit, which is suitable or equipped for the detection of the specific features of the insects located within the defined compartment interior or within the insect trap is formed by optical detection devices or comprises optical detection devices. Optical detection devices in the present context are intended to mean any sensor technology capable of detecting electromagnetic waves or changes in the emitting behavior or reflecting behavior for electromagnetic waves in the infrared range, in the visible range, and/or in the ultraviolet range. In this connection, the optical detection devices can comprise, in particular, an image evaluation disposed downstream from the sensor unit, in which context the detection of reflective elements or reflective image parts can be expedient. Optionally, movement patterns of the sensed insects can be detected and/or provided to the downstream electronic analysis device and/or evaluation device by the optical detection devices and/or by the downstream image evaluation. Any other detection variants are also generally conceivable, for example, the optical detection of acoustic patterns emitted by the insects in question, or of wing-beat profiles that can be characteristic of the insects in question. Other emissions, such as chemical material compositions or the like, can also be detected. If optical detection of acoustic patterns is referred to in this context, this is intended to mean so-called opto-acoustic methods, which can, for example by laser detection methods, detect typical acoustic emissions and emission patterns generated by particular insects in a different manner, such that a reliable distinction of different insect species and also a reliable distinction of male and female insects of the same species is made possible based on such a pattern detection and a downstream evaluation of the emissions detected in such a way.

If the insects are distinguishable and/or identifiable on the basis of their acoustic emissions, the method can optionally also provide that the sensor unit, which is suitable or equipped for the detection of the specific features of the insects located within the defined compartment interior or within the insect trap is formed by acoustic detection devices or comprises acoustic detection devices. Further variants of the method are conceivable, for example, that the sensor unit, which is suitable or equipped for the detection of the specific features of the insects located within the defined compartment interior or within the insect trap is formed by detection devices that are sensitive to electromagnetic and/or radioactive radiation or comprises detection devices that are sensitive to electromagnetic and/or radioactive radiation.

The method can provide that the insects attracted within the defined spatial vicinity or in the vicinity of an insect trap are attracted by optical attracting stimuli that are attractive to the insects or flying insects, for example, by light/dark contrast surfaces or the like. Optionally, the method can also provide that the insects attracted within the defined spatial vicinity or in the vicinity of an insect trap are attracted by acoustic stimuli that are attractive to the insects or flying insects. The use of other stimuli is also possible, and likewise the combination of a plurality of these attracting stimuli. Optionally, the method can thus also provide that the insects attracted within the defined spatial vicinity or in the vicinity of an insect trap are attracted by sensory stimuli that are attractive to the insects and/or by heat. The method can likewise be designed such that the insects attracted within the defined spatial vicinity or in the vicinity of an insect trap are attracted by olfactory and/or chemical attracting stimuli that are attractive to the insects or flying insects.

As a further alternative or addition, a variant of the method according to the invention can be designed such that the insects attracted within the defined spatial vicinity or in the vicinity of an insect trap are attracted by light stimuli that are attractive to the insects and/or by acoustic stimuli that are attractive to the insects or flying insects. It has thus been shown that mosquitoes and other flying insects are attracted by light stimuli and that male mosquitoes, in particular, are attracted by ultraviolet light, so that they perceive this ultraviolet light as a distinct attracting stimulus. It has moreover been shown that a combination of light stimuli, in particular using UV light, with acoustic stimuli is perceived as particularly attractive to the male insects. In particular, these acoustic stimuli can be formed by an imitation of wing beats of female insects, which are perceived as a strong attracting stimulus by the male insects.

All other types of suction devices and/or elements of suction traps should generally be regarded as attractants, too, because it is also generally possible to suck in the insects in a suitable manner without presenting them with specific attracting stimuli.

In order to prevent double counts of the same insects, it can be provided in the method that the attracted insects within the defined compartment interior or in the interior space of the insect trap are prevented from escape for a defined time interval. Thus, the method can be designed such that the insects attracted within the defined compartment interior or in the interior space of the insect trap are released after having been detected. The release after the detection can be important and expedient, in particular, if sterile male insects are released, as they can continue to swarm out into the examined vicinity after having been detected and released.

As an alternative, the method can also be altered in such a manner that the attracted insects within the defined compartment interior or in the interior space of the insect trap are prevented from escape for an indefinite period of time. That is to say that the insects can be captured, detected, and subsequently rendered harmless or be killed. This is another way of ensuring that the captured insects are in each instance only detected once.

Methods based on detecting and measuring the insects that are carried in an airflow, for example, are suitable for the detection of the insects captured inside the insect traps. Since these methods, however, are relatively difficult to handle in practice, variants are also possible in which the insects are fixated on a defined surface, for example, and detected in that position by the sensor unit. Such a defined surface can be, for example, an adhesive surface or a net or the like, located within the trap to reliably prevent multiple measurements.

It is furthermore possible with the method to detect the sensor data from at least one insect trap equipped with appropriate sensor technology over a defined, longer period of time and to detect, analyze, and prepare the sensor data for the analysis of populations of insects and/or of their changes over the course of time. Preferably, however, the data from a plurality of such traps is used for such an analysis method. The electronic analysis device and/or evaluation device, which is disposed downstream from the particular sensor unit of each individual trap of a total of several such insect traps that are potentially each situated in a different location, can communicate, in particular, via remote data connections with the insect traps or with their sensor units. So that a central data detection and data evaluation for a larger area that is provided with a plurality of traps, potentially with a multitude of insect traps, is made possible without a direct line connection or data connection being required.

Insects of a certain species or genus, for example mosquitoes or mosquitoes of a certain genus, can be detected and monitored with the method according to the invention. In this way it is possible to gain insights about the ratio between the natural population and released mosquitoes or also data on the population in general. It is possible, for example, to verify the survival time, the distribution, the incidence, etc., of released mosquitoes. A central aspect of the method, however, lies in distinguishing certain insects, that is to say in particular, in distinguishing marked from non-marked insects.

To achieve the above-mentioned goal, the invention furthermore proposes an insect detection system and/or insect monitoring system used for the detection and/or monitoring of insects of at least one defined genus, which insects are attracted and at least temporarily retained within a defined compartment, and which are sensed and/or analyzed with regard to definable features, the system having the features of the independent system claim. The system at least comprises suitable agents to attract (called attractants) the defined genus of insects within a definable spatial vicinity by attracting stimuli that are attractive to the insects or flying insects, as well as a defined compartment interior, formed, in particular, by an interior space of an insect trap, which is provided and/or equipped for the transfer and accommodation of the attracted insects or flying insects, while preventing, at least for a definable time interval, an inadvertent escape, at least of a large number of the insects having been transferred into the defined compartment interior. The system furthermore requires a suitable sensor unit, which is suitable or equipped for the detection of specific features of the insects located within the defined compartment interior or within the insect trap and an electronic analysis device and/or evaluation device, which is disposed downstream from the sensor unit, for the detection and/or evaluation of the specific features of the insects detected by the sensor unit.

The system according to the invention can be designed, in particular, for the detection of markers adhering to the insects and/or of markers having been applied to them by stamping. The sensor unit, which is suitable or equipped for the detection of the specific features of the insects located within the defined compartment interior or within the insect trap can be formed by optical detection devices, for example, or can comprise such optical detection devices. The optical detection devices can thus comprise, for example, an image evaluation disposed downstream from the sensor unit, in which context devices for the detection of reflective elements or reflective image parts can also be expediently used. Optionally, movement patterns of the sensed insects can be detected and/or provided to the downstream electronic analysis device and/or evaluation device by the optical detection devices and/or by the downstream image evaluation. Also conceivable are variants in which various other features are detected, for example, acoustic patterns, wing beat profiles, other emissions from the insects, etc. Thus, the sensor unit, which is suitable or equipped for the detection of the specific features of the insects located within the defined compartment interior or within the insect trap can in this system be formed by acoustic detection devices or can comprise such acoustic detection devices.

The system can furthermore be equipped in such a manner that the sensor unit, which is suitable or equipped for the detection of the specific features of the insects located within the defined compartment interior or within the insect trap is formed by detection devices that are sensitive to electromagnetic and/or radioactive radiation or comprises detection devices that are sensitive to electromagnetic and/or radioactive radiation.

The system can optionally be capable of using optical and/or acoustic and/or sensory stimuli that are attractive to the insects and/or heat stimuli and/or olfactory and/or chemical attracting stimuli by which the insects are attractable, in which context it is also possible to employ and apply any combinations of these variants of attracting stimuli. If optical and/or acoustic attracting stimuli are referred to, it should be particularly pointed out that mosquitoes and other flying insects are attracted by light, and that male mosquitoes, in particular, are attracted by ultraviolet light and perceive this ultraviolet light as a distinct attracting stimulus. It has moreover been shown that a combination of light stimuli, in particular using UV light with acoustic stimuli, is perceived as particularly attractive to the male insects. In particular, these acoustic stimuli can be formed by an imitation of wing beats of female insects, which are perceived as a strong attracting stimulus by the male insects.

The defined compartment interior or the interior space of the insect trap which is provided for the accommodation of attracted insects or flying insects, can prevent the accommodated insects at least for a defined time interval from an escape. Optionally, the insects can also be rendered harmless after having been detected and after the detected data having been evaluated. The employed insect traps can be equipped, in particular, with a device for the fixation of the captured insects or flying insects. Such a device or a defined surface in the trap, can be an adhesive surface or a net or the like, which is located inside the trap and in the detection range of the employed sensor unit, whereby multiple measurements are reliably prevented. Methods based on detecting and measuring the insects that are carried in an airflow, for example, are suitable for the detection of the insects captured inside the insect traps, in which context it should be mentioned that in practice such methods require more input than the type of methods in which the insects are fixated and detected in that position by the sensor unit.

The system can be used or employed, in particular, to perform a method according to one of the above-described embodiment variants.

It should be explicitly mentioned at this point that all aspects and embodiment variants explained in the context of the insect detection system and/or insect monitoring system according to the invention can likewise pertain to or constitute partial aspects of the method according to the invention. If specific aspects and/or interrelations and/or effects relating to the insect detection system and/or insect monitoring system according to the invention are referred to at some point in the present description or in the claims definitions, this therefore likewise pertains to the method according to the invention. The same applies conversely, so that all aspects and embodiment variants explained in the context of the method according to the invention can likewise pertain to or constitute partial aspects of the insect detection system and/or insect monitoring system according to the invention. If specific aspects and/or interrelations and/or effects relating to the method according to the invention are referred to at some point in the present description or in the claims definitions, this therefore likewise pertains to the insect detection system and/or insect monitoring system according to the invention.

### BRIEF DESCRIPTION OF THE FIGURES

In the following passages, the attached figures further illustrate typical embodiments of the invention and their advantages. The size ratios of the individual elements in the figures do not necessarily reflect the real size ratios. It is to be understood that in some instances various aspects of the invention may be shown exaggerated or enlarged in relation to other elements to facilitate an understanding of the invention.

FIG. 1 shows a schematic illustration of an embodiment variant of an insect detection system and/or insect monitoring system according to the invention.

FIG. 2 shows a schematic illustration of the active principles of the system according to FIG. 1, which system is provided or suitable for carrying out a method according to the invention, the method being used for the detection and/or monitoring of insect populations of a defined genus.

The same or equivalent elements of the invention are designated using identical reference characters. Furthermore and for the sake of clarity, only the reference characters relevant for describing the individual figures are provided. It should be understood that the detailed description and specific example of the optical monitoring system according to the invention, while indicating a preferred embodiment, is intended for purposes of illustration only and is not intended to limit the scope of the invention.

### DETAILED DESCRIPTION OF THE INVENTION

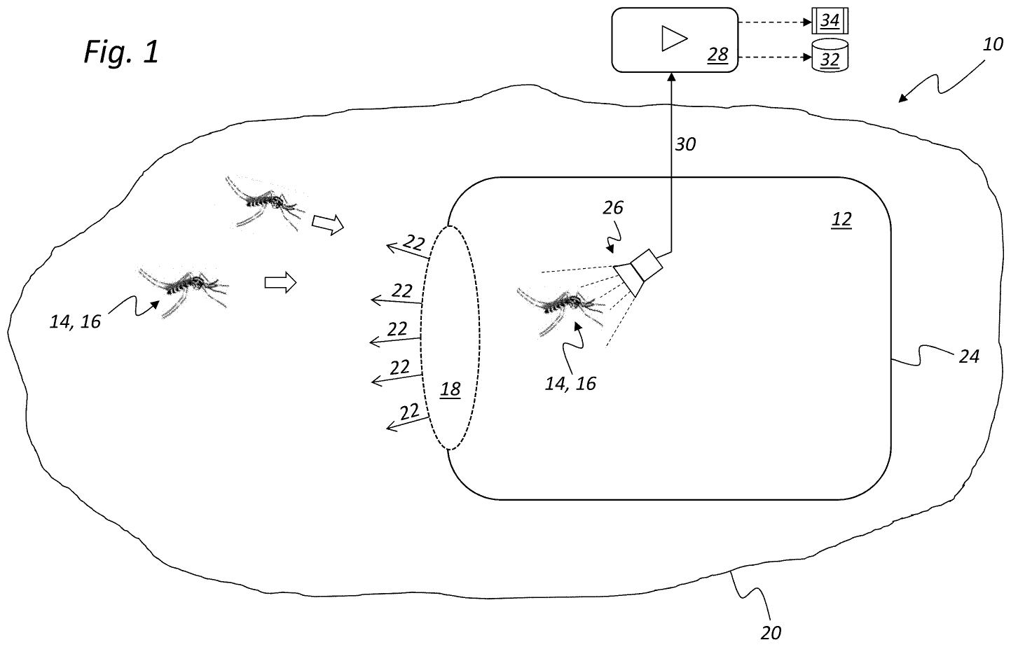

The schematic illustration of FIG. 1 shows the essential principles of a possible embodiment variant of an insect detection system and/or insect monitoring system 10 or of a system 10 used for the detection and/or monitoring of insects 16 of at least one defined genus, which insects 16 are attracted and at least temporarily retained within a defined compartment 12, and which are sensed and/or analyzed with regard to definable features 14. The flying insects 16 can be yellow-fever mosquitoes (Aedes aegypti), for example, which occur in great numbers in some areas, and of which the population can be monitored by the shown system 10, provided that the insects 16 located and detected within a defined compartment 12 have a uniquely sensable marker representing the definable features 14. The flying insects 16 in the sense of the present invention can also be nearly any other species, as the principles according to the invention can be used for most diverse insect species.

The system 10 comprises attractants 18—which are not described in more detail here to begin with—to attract the defined genus of insects 16 within a definable spatial vicinity 20 within which the attractants 18 are effective and have an attracting influence on those insects 16 that are present within the definable spatial vicinity 20. The attractants 18 can emit, in particular, attracting stimuli 22 that are attractive to the insects 16 and that can signalize them optical, acoustic, olfactory, or other attracting stimuli 22, for example those of an attractive food source. The defined compartment interior 12 to which the attractants 18 to attract the flying insects 16 are assigned, can be formed, in particular, by an interior space of an insect trap 24, which serves for the transfer and accommodation of the attracted insects 16, while preventing, at least for a definable time interval, an inadvertent escape, at least of a large number of the insects 16 having been transferred into the defined compartment interior 12.

The insect traps 24 employed in this connection can be of various different types according to the insects 16 to be attracted and captured. For yellow-fever mosquitoes or mosquitoes, for example, such traps have proved particularly effective that combine optical contrast surfaces with weak airflows, which are perceived as distinct attracting stimuli by the relevant insects. WO 2004 054 358 A2, for example, discloses such an insect trap 24. A light surface is combined there with a diffuser surface to generate a weak airflow emanating from the surface, in which context at least one dark contrast spot lies within the light surface. The dark contrast spot can be formed, in particular, as intake suction duct to suck in the attracted flying insects 16.

In order to be able to fulfill its intended object, the system 10 furthermore comprises a suitable sensor unit 26 disposed, in particular, within the compartment interior 12 of the trap 24, which system 10 is suitable and equipped for the detection of the previously defined, specific features 14 of the insects 16 located within the defined compartment interior or insect trap 24. It should be pointed out, however, that the sensor unit 26 by no means necessarily has to be spatially assigned to the trap 24 in such a manner that it is located in the insect trap 24. The sensor unit 26 can likewise be disposed merely in spatial vicinity to the defined compartment interior 12 or to the insect trap 24. Thus, arrangements have proved successful in which, for example, the trap 24 is monitored by optical detection devices from above, with the sensor unit 26 of the optical detection devices employed in this context not being an immediate component of the insect trap 24. Furthermore, an electronic analysis device and/or evaluation device 28 for the detection and/or evaluation of the specific features 14 of the insects 16 detected by the sensor unit 26 is disposed downstream from the sensor unit 26, which electronic analysis device and/or evaluation device 28 can process and prepare in a suitable manner the electronic sensor signals 30 supplied by the sensor unit 26. Preferably, the electronic analysis device and/or evaluation device 28 can store and/or visualize or transmit to remote locations in a suitable manner the data obtained and calculated from the sensor signals 30 that are supplied from the processing and preparing by the sensor unit 26, and this is indicated by the storage device 32, which is coupled with the analysis device and/or evaluation device 28 and which is to be regarded as a further equipment option, as well as by the display device 34, which is likewise coupled with the analysis device and/or evaluation device 28 and likewise to be regarded as a further equipment option.

If a device 28 disposed downstream from the sensor unit 26 is referred to in the present context, this is to be understood in a general and comprehensive sense. For example, numerous sensor units 26 can transmit their data 30 to a central analysis device and/or evaluation device 28 without a spatial proximity being necessary in this context between the traps 24 and the device 28. The system 10 according to the invention can also comprise a network of a plurality of or of many insect traps 24 that cover a larger area, for example. The sensor data 30 supplied by the sensor units 26 from the a plurality of or from the numerous insect traps 24 can in this context be supplied, for example, by remote connections (e.g. radio connections) to the central data processing equipment 28, which can optionally be located remote from the traps 24. The electronic analysis device and/or evaluation device 28, which is disposed downstream from the particular sensor unit 26 of each individual trap 24 of a total of several such insect traps 24 that are potentially each situated in a different location, can communicate, in particular, via remote data connections with the insect traps 24 or with their sensor units 26 so that a central data detection and data evaluation for a larger area that is provided with a plurality of traps 24, potentially with a multitude of insect traps 24, is made possible without a direct line connection or data connection being required. In this way it is possible to realize high-performance monitoring systems for large areas, which can be centrally administered and equipped with a central data detection and data evaluation.

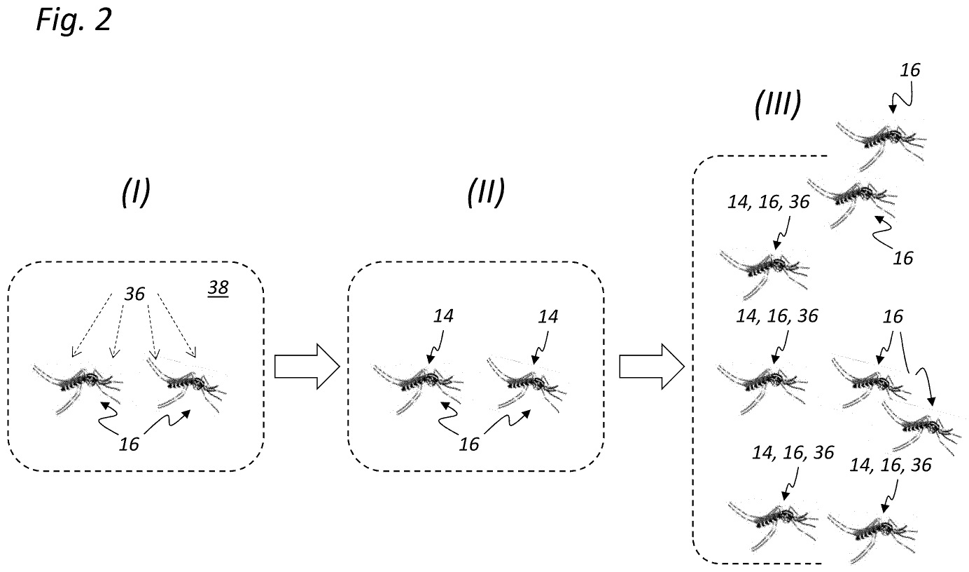

The schematic presentation of FIG. 2 is intended to illustrate the principles forming the basis of the system 10 according to the invention as explained in FIG. 1. The method described with the present invention is primarily a measure to monitor effectiveness, that is to say, it is intended to verify and potentially accompany and monitor the effectivity and range of manipulation measures over longer periods of time. If specific features 14 are referred to in the present context, with the features 14 adhering to the flying insects 16 attracted and detected by the sensor unit 26, these features 14 can be, for example, colored, fluorescent, or other optically identifiable markers 36 administered to or applied onto the flying insects 16 bred in a laboratory 38. These laboratory-38-bred flying insects (e.g. yellow-fever mosquitoes/Aedes aegypti or the like) are sterile male animals, which after their release would no longer be distinguishable from wild animals without the sensable features 14 applied onto them in the form of the markers 36. After having bred the animals 16 in the laboratory 38 and applied the markers 36 (phase I, left box), they are releasable male and sterile flying insects 16 that are provided with distinctive and uniquely sensable features 14 (phase II, next box to the right). The insects 16 can be administered the markers 36 by suitable feeding, for example, and be imprinted by being fed with a low-concentration solution of the fluorescent substance rhodamine B, which can be identified on the insects 16 in the desired manner by optical sensors.

The in such a way marked, laboratory-38-bred, sterile, male flying insects 16 with the markers 36 applied onto them can subsequently be released within a population of wild flying insects 16—these are female and male animals without features or markers applied onto them—as is indicated by the box on the right side in FIG. 2 (phase III). Since the laboratory-38-bred animals stay in the existing population and mate with female animals, due to their sterility, however, prevent reproduction of the animals, the existing wild population of the flying insects 16 manipulated in such a way is gradually reduced, if the insects behave as desired and predicted.

However, neither is the population density of an existing population of wild flying insects 16 precisely determinable, nor is the influence on the population by the release of the marked sterile insects 16 verifiable by mere observation, so that the system 10 described in a schematic way in FIG. 1 can serve to detect and document the population changes by the marked insects 16 being uniquely detectable and distinguishable from non-marked, that is to say, wild insects 16.

The sensor unit 26, which is shown in FIG. 1, and which is suitable or equipped for the detection of the specific features 14 of the insects 16 16 located within the defined compartment interior 12 or within the insect trap 24 can be formed, for example, by optical detection devices, such as cameras, or can comprise such optical detection devices. The optical detection devices can thus comprise, for example, an image evaluation step or device disposed downstream from the sensor unit 26 (in particular within the electronic analysis device and/or evaluation device 28), in which context devices for the detection of reflective elements or reflective image parts can also be expediently used. Optionally, movement patterns of the sensed insects 16 can be detected and/or provided to the downstream electronic analysis device and/or evaluation device 28 by the optical detection devices and/or by the downstream image evaluation. Also conceivable are variants in which various other features are detected, for example, acoustic patterns, wing beat profiles, other emissions from the insects 16, etc. Thus, the sensor unit 26, which is suitable or equipped for the detection of the specific features 14 of the insects 16 16 located within the defined compartment interior 12 or within the insect trap 24 can in this system 10 also be formed by acoustic detection devices or can comprise such acoustic detection devices. The system 10 can furthermore be equipped in such a manner that the sensor unit 26, which is suitable or equipped for the detection of the specific features 14 of the insects 16 located within the defined compartment interior 12 or within the insect trap 24 is formed by detection devices that are sensitive to electromagnetic and/or radioactive radiation or comprises detection devices that are sensitive to electromagnetic and/or radioactive radiation.

The attractants 18 indicated in FIG. 1 can emit various different attracting stimuli 22, for example optical and/or acoustic and/or sensory stimuli that are attractive to the insects 16 and/or heat stimuli and/or olfactory and/or chemical attracting stimuli by which the insects 16 are attractable, in which context it is also possible to employ and apply any combinations of these variants of attracting stimuli.

The defined compartment interior 12 or the interior space of the insect trap 24 which is provided for the accommodation of attracted insects 16, can prevent the accommodated insects 16 at least for a defined time interval from an escape. Optionally, the insects 16 can also be rendered harmless or be killed after they have been detected and after the detected data has been evaluated.

The invention has been described with reference to a preferred embodiment. Those skilled in the art will appreciate that numerous changes and modifications can be made to the preferred embodiments of the invention and that such changes and modifications can be made without departing from the spirit of the invention. It is therefore intended that the appended claims cover all such equivalent variations as fall within the true spirit and scope of the invention.

### LIST OF REFERENCE CHARACTERS

10 System, insect detection system and/or insect monitoring system

12 Compartment, defined compartment

14 Feature, definable features

16 Insects, flying insects, mosquitoes, yellow-fever mosquitoes

18 Attractant, agent to attract insects

20 Spatial vicinity, definable spatial vicinity

22 Attracting stimulus, stimuli

24 Trap, insect traps

26 Sensor unit, sensor technology unit, sensor technology for the detection of insect features

28 Analysis device and/or evaluation device, electronic analysis device and/or evaluation device

30 Output signal, sensor signal, sensor data

32 Storage device

34 Display device, display

36 Marker, optical marker

38 Laboratory

We claim:

1. A method used for the detection and/or monitoring of insect populations (16), comprising:
attracting a defined genus of insects (16) into a spatial vicinity (20) of a defined compartment interior (12) with an attracting stimuli (22);
transferring the attracted insects (16) into the defined compartment interior (12) while preventing, at least for a time interval, an inadvertent escape of a large number of the insects (16) that have been transferred into the defined compartment interior (12);
detecting specific features (14) of the insects (16) located within the defined compartment interior (12) by a sensor unit (26);
transmitting sensor signals (30), representing the detected specific features (14) to an evaluation device (28) disposed downstream from the sensor unit (26).

2. The method of claim 1, wherein the detected specific features (14) of the insects (16) are sex, species, or genus of the insects (16).

3. The method of claim 1, wherein the detected specific features (14) of the insects (16) are markers (36) borne by the insects (16) or are markers (36) having been applied to the insects (16).

4. The method of claim 3, wherein the markers (36) are formed by genetic modifications to the insects (16). genetic modifications are detected by means of the sensor technology (26).

5. The method claim 3, wherein the markers (36) are radioactive markers.

6. The method of claim 3, wherein the markers (36) are optical or fluorescent markers.

7. The method of claim 3, wherein the markers (36) are formed by administering a substance to the insects (16).

8. The method of claim 1, wherein the sensor unit (26) comprises one or more optical detection devices.

9. The method of claim 8, wherein the one or more optical detection devices comprises an image evaluation device.

10. The method of claim 8, wherein the detecting step comprises detecting movement patterns of the insects (16).

11. The method of claim 1, wherein the sensor unit (26) comprises one or more acoustic detection devices.

12. The method of claim 1, wherein the sensor unit (26) comprises one or more detection devices that are sensitive to electromagnetic or radioactive radiation.

13. The method of claim 1, wherein the attracting stimuli (22) is an optical attracting stimuli (22).

14. (canceled)

15. The method of claim 13, wherein the optical attracting stimuli (22) are substantially formed by the emission of UV light or contain UV light components.

16. The method of claim 1, wherein the attracting stimuli (22) is an acoustic stimuli (22).

17. The method of claim 1, wherein the attracting stimuli (22) is a heat, olfactory, or chemical stimuli (22).

18. (canceled)

19. (canceled)

20. (canceled)

21. The method of claim 1, further comprising releasing the insects (16) from the defined compartment interior (12) after the detecting step.

22. (canceled)

23. The method of claim 1 wherein the detecting step comprises holding the insects (16) in an airflow generated within the defined compartment interior (12).

24. The method of claim 1, wherein the detecting step comprises holding the insects (16) on an adhesive surface or on a net.

25. The method of claim 1, further comprising analyzing changes in populations of insects (16) over a period of time.

26-38. (canceled)
